(19) United States
(12) Patent Application Publication
(10) Pub. No.: US 2004/0115232 A1
(43) Pub. Date: Jun. 17, 2004

(54) Cosmetic composition for volumizing keratin fibers and cosmetic use of nanotubes for volumizing keratin fibers

(76) Inventors: Franck Giroud, Clichy (FR); Valerie Favreau, Taverny (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/455,499

(22) Filed: Jun. 6, 2003

(30) Foreign Application Priority Data
Jun. 6, 2002 (FR) 02 06967

(51) Int. Cl. A61K 7/021; A61K 7/00

(52) U.S. Cl. 424/401; 424/63

(57) ABSTRACT

A cosmetic composition for volumizing keratin fibers, comprising, in a cosmetically acceptable medium, certain nanotubes, and the cosmetic use of these nanotubes for volumizing keratin fibers.

Disclosed herein are novel compositions for volumizing keratin fibers, for example, hair, comprising nanotubes, and the cosmetic use of nanotubes for volumizing keratin fibers.

Many styling products exist for volumizing the hair. A drawback that can be associated with these products, which are usually based on film-forming polymers, may lie in the fact that the cosmetic effect can disappear with the first shampoo wash.

Permanent-waving treatments of keratin fibers are moreover known. These treatments can use a reducing agent and an oxidizing agent, and may require the hair to be placed under mechanical tension using rolling equipment, so as to give the hair a long- lasting shape.

These processes, which can effectively make it possible to increase the volume of the hair, can have the drawback, however, of modifying the level of curliness of the hair and of degrading the feel of the fibers.

It thus appears to be necessary to develop compositions that can increase the volume of the hair without modifying the shape or feel of the hair.

Disclosed herein are novel compositions for remedying at least one of these drawbacks.

The inventors have surprisingly discovered that a composition comprising nanotubes can give the hairstyle volume, and may not adversely affect the feel of the hair or its shape, degrade the fibers and make the hairs adhere together with a film-forming material. In addition, these cosmetic properties can be shampoo-resistant.

Disclosed herein is thus a cosmetic composition for volumizing keratin fibers, comprising nanotubes.

Further disclosed herein is the cosmetic use of nanotubes for volumizing keratin fibers.

Other embodiments disclosed herein will become apparent on reading the description and the examples that follow, without, however, being limiting in nature.

The cosmetic compositions disclosed herein comprise, in a cosmetically acceptable medium, nanotubes comprising at least one element chosen from elements of groups II_{A}, III_{A}, IV_{A}, V_{A}, VIII, I_{B}, II_{B}, III_{B}, VI_{B} and VII_{B} of the Periodic Table of the elements.

As used herein, the term “nanotubes” means nano-objects whose atomic or molecular organization gives the nanostructure a tubular shape. These nanotubes may be single-walled or multi-walled.

The diameter of the nanotubes can range, for example, from 1 to 300 nm and the length of the nanotubes can range, for example, from 10 nm to 10 mm.

The at least one element of the nanotubes disclosed herein may, for example, be chosen from carbon, silicon, tungsten, silver, gold, boron, zinc, platinum, magnesium, iron, cerium and aluminium.

For example, the nanotubes can comprise at least one element chosen from elements of group IV_{A} of the Periodic Table of the elements. Further, for example, the at least one element can be carbon.

When the at least one element of the nanotubes is carbon, said nanotubes may, for example, comprise, totally or partially, organic molecules. The organic molecules may, for example, be chosen from diacetylenic phospholipids, glutamates, long-chain diamides, glucophospholipids and alkylphenylglucopyranosides.

Further, the skeleton of the nanotubes may comprise, for example, solely carbon atoms.

The attached FIGS. 1 to 6 show different carbon nanotubes used according to the embodiments disclosed herein.

These carbon nanotubes may conventionally be obtained by sublimating graphite at a very high temperature using an electric arc. The carbon nanotubes may be; formed by a single plane of graphene; in this case, they are referred to as single-wall nanotubes (SWNT). FIG. 1 shows a single-wall nanotube. The graphene planes may be rolled up in a form chosen from zig-zag, slot and chiral forms. The carbon nanotubes may also, for example, comprise several tubes “fitted” into each other; in this case, they are referred to as multi-wall nanotubes (MWNT).

In one embodiment disclosed herein, in order to obtain optimum solubilization or exfoliation of the carbon nanotubes in the cosmetic medium under consideration, the surface of the nanotubes may, for example, be functionalized.

As used herein, the term “functionalized” means the presence of at least one functional group that can physically or chemically interact with a keratin material or with the external medium.

Any reaction mechanism may, for example, be used to functionalize the graphene planes constituting the carbon nanotubes. For example, the carbon nanotubes may be functionalized via at least one reaction mechanism chosen, for example, from nucleophilic substitution, electrophilic substitution, free-radical substitution, addition, elimination, rearrangement, oxidation, reduction, acid-base reaction, electrochemical reaction and photochemical reaction.

The at least one functional group that may, for example, be grafted onto the surface of the graphene planes constituting the carbon nanotubes, may, for example, be chosen from carboxylic groups. FIG. 2 shows a single-wall nanotube at the surface of which are grafted carboxylic groups. This functionalization is described, for example, in the article “Solution Properties of Single Walled Carbone Nanotube” J. Chen et al. (Science 1998, vol. 282, No. 5396, pages 95-98).

The carbon nanotubes may, for example, be solubilized in a polar solvent, for example, a polar solvent chosen from water and ethanol, by oxidizing the graphene planes with a HCl/CrO₃ mixture, described in the article “Room Temperature Filling of Single Wall Carbon Nanotubes With Oxide In Open Air”, J. Mittal et al. (Chem. Phys. Lett. 2001, vol. 339, No. 5-6, pages 311-318) or by condensing an amino acid and an aldehyde onto the nanotubes (J. Am. Soc., vol. 124, No. 5, 2002, pages 760 and 761).

Hydrophobic functional groups may also be grafted, for example, onto the surface of the graphene planes constituting the carbon nanotubes. Mention may be made, for example, of the fluorination of carbon nanotubes, described in the article “Fluorinated Single Wall Nanotubes”, K. N. Kudin et al. (Phys. Rev. 2001, B63, 45413).

Mineral molecules such as alkoxysilanes may also be grafted onto the surface of the graphene planes constituting the carbon nanotubes. See Nano. Lett., vol..2, No. 4, 2002, pages 329 to 332.

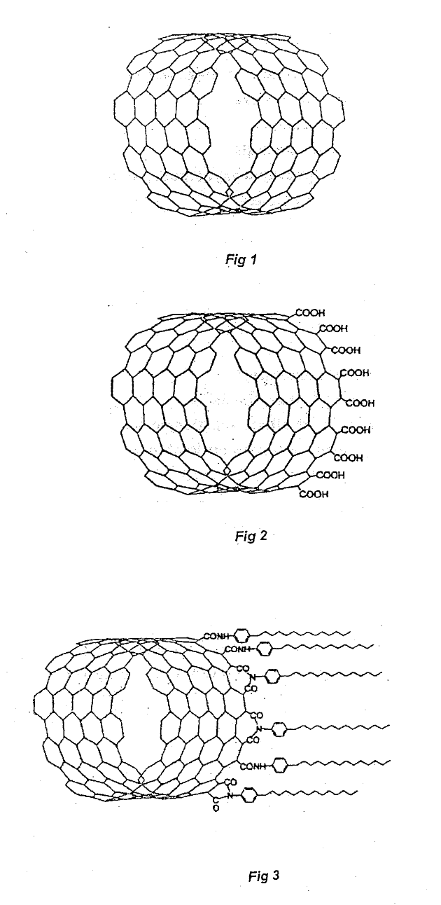

Graphene planes may be functionalized in several steps as in, for example, the functionalization of carbon nanotubes with fatty-chain amides, described in the article “Dissolution of Single Wall Carbon Nanotube”, M. A. Hamon et al. (Adv. Mater. 1999, vol. 11, No.10). FIG. 3 shows a single-wall carbon nanotube whose surface is functionalized with the fatty-chain amide functional group,

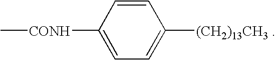

This multi-step functionalization may also lead, for example, to the grafting of glucose. See Nano. Lett., vol. 2, No. 4, 2002, pages 369 to 373.

The functionalization of carbon nanotubes may, for example, be performed with simple molecules, but also, for example, can be performed with oligomers, polymers or dendrimers. Mention may be made, for example, of the article “A New Purification Method for Single Wall Carbon Nanotubes”, M. Holzinger et al. (Appl. Phys. A 70 (2000) 599), which describes the grafting of dendritic structures onto the surface of graphene planes constituting the carbon nanotube. FIG. 4 shows a single-wall carbon nanotube whose surface is functionalized with dendritic structures.

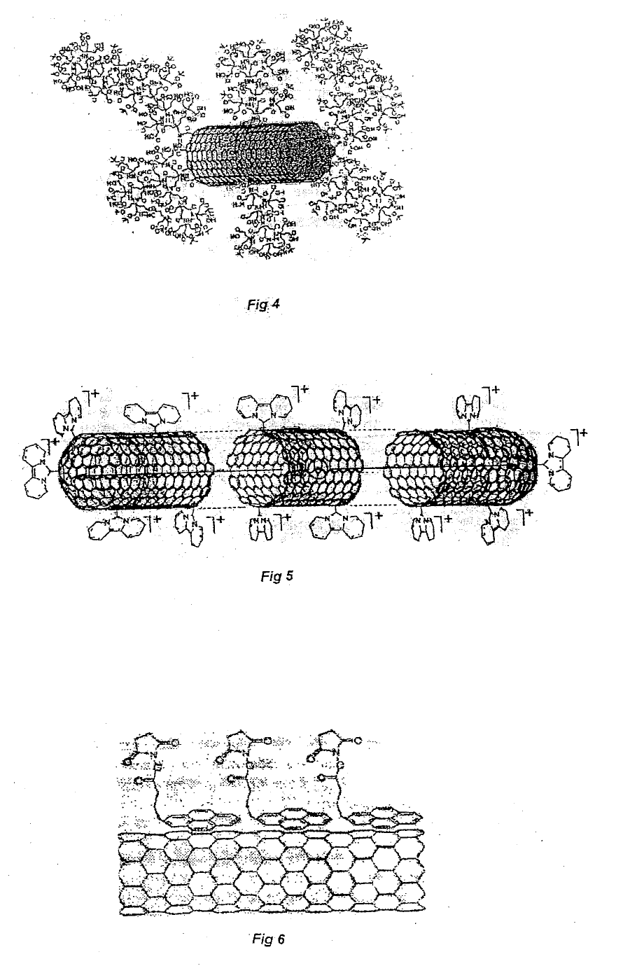

Besides improving the dispersion of the carbon nanotubes in a cosmetic medium, functionalization of the surface may also, for example, be performed in order to increase the affinity of the carbon-based nanostructures for a keratin material. Improving the affinity between the nanotubes and the keratin material, induced by functionalizing the graphene planes, may be the fruit of increasing the interactions of Van der Waals type and/or the fruit of the appearance of hydrogen bonding and/or ionic bonds. Thus, the at least one functional group can be capable of creating with keratin fibers at least one chemical bond chosen from interactions of Van der Waals type, hydrogen bonding, ionic bonds and covalent bonds. In this context, mention may be made of the grafting of cationic molecules onto the surface of carbon nanotubes, described in the article “Exohedral Sidewall Reactions of Single Walled Carbon Nanotubes in Molecular Nanostructures”, M. Holzinger et al. (Proceedings of the XIIth International Winterschool on Electronic Properties of Novel Materials: Molecular Nanostructures, Kirchberg, Austria, March 2001). In the context of grafting cationic molecules, mention may also be made of the grafting of polyethyleneimine derivatives. See Nano. Lett., vol. 2, No. 3, 2002, pages 231 to 234. FIG. 5 shows a single-wall carbon nanotube whose surface is functionalized with cationic molecules.

To increase the affinity of carbon-based nanotubes for the keratin material, such as keratin fibers, it is also possible to covalently graft the carbon-based nanotubes onto the keratin material. To do this, the functionalization of the graphene planes is performed using at least one group that has a certain level of reactivity on the amino acids constituting the keratin material. For example, the at least one functional group capable of creating at least one covalent chemical bond with the keratin fibers is chosen from groups capable of reacting with thiols, disulphides, carboxylic acids, alcohols and amines. For example, the at least one functional group capable of reacting with thiols, disulphides, carboxylic acids, alcohols and amines is chosen from:

epoxides,

groups comprising at least one aziridine ring,

vinyl and activated-vinyl groups, such as acrylonitrile, acrylic and methacrylic esters, crotonic acid and ester, cinnamic acids and esters, styrene and derivatives, butadiene, vinyl ethers, vinyl ketone, maleic esters, maleimides, vinyl sulphones and vinyl cyanoacrylate,

carboxylic acids and derivatives thereof, for example, anhydride, acid chloride and ester functional groups,

acetals and hemiacetals,

aminals and hemiaminals,

ketones, α-hydroxy ketones and α-halo ketones,

lactones and thiolactones,

isocyanate,

thiocyanate,

imines,

imides (succinimides and glutimides), such as N-hydroxysuccinimide ester,

imidates,

oxazine and oxazoline,

oxazinium and oxazolinium,

alkyl, aryl, and aralkyl halides, wherein the halogen is chosen from iodine, bromine and chlorine,

unsaturated-ring halides, wherein the unsaturated rings are chosen from carbon-based rings and heterocycles such as chlorotriazine, chloropyrimidine, chloroquinoxaline and chlorobenzotriazole,

sulphonyl halides of formula RSO₂X, wherein R is chosen from alkyl groups and X is chosen from fluorine and chlorine, and

silicon derivatives such as alkoxysilanes and silanols.

In order to obtain optimum dispersion (exfoliation) of the carbon nanotubes in the cosmetic medium under consideration, the composition may comprise, for example, at least one surfactant. The at least one surfactant may, for example, be chosen from amphiphilic molecules, amphiphilic oligomers, amphiphilic dendrimers and amphiphilic polymers.

The at least one surfactant may comprise, for example, at least one hydrophobic group, wherein the at least one hydrophobic group may, for example, be chosen from groups capable of being absorbed by “Ti-stacking” (overlapping of the electron clouds of the aromatic nuclei) at the surface of the graphene planes constituting the carbon nanotubes. Such groups may, for example, be chosen from aromatic molecules such as styrene and derivatives thereof and pyrene and derivatives thereof.

The at least one surfactant may also, for example, comprise hydrophilic groups and/or blocks, wherein the hydrophilic groups and/or blocks may be chosen from nonionic, amphoteric, zwitterionic, anionic and cationic groups and/or blocks.

The hydrophilic groups and/or blocks may, for example, be chosen so as to improve the affinity of the surface of the nanotubes for the keratin material, either by increasing the interactions of Van der Waals type and/or creating hydrogen bonding and/or ionic bonds. The hydrophilic groups may, for example, be chosen from groups of cationic nature.

In order to increase the affinity of the nanotubes for the keratin material, the at least one surfactant may also be capable of creating with the keratin fibers at least one covalent chemical bond, for example, by reaction on the disulphide, thiol, carboxylic acid, amine and alcohol functional groups of the amino acids of which the keratin material is composed. One example that may be mentioned is the use of reactive surfactant systems of the pyrene succinimidyl ester type described in the article “Noncovalent Sidewall Functionalization of Single Walled Carbon Nanotubes for Protein Immobilization”, J. Chen et al. (J. Am. Chem. Soc., 123, (16), 3838-3839, 2001). The carbon nanotubes may thus be stabilized with 1-pyrenebutanoic acid succinimidyl ester. The succinimidyl ester functional group is capable of reacting at room temperature with the amine functional groups of the keratin material. FIG. 6 shows a single-wall carbon nanotube whose surface is functionalized with 1-pyrenebutanoic acid succinimidyl ester.

Certain saccharides, for example, cyclic saccharides, such as cyclodextrins, also may have excellent dispersing power with respect to carbon-based nanostructures. See J. Am. Chem. Soc., vol. 123, 6201 (2001). Hyperbranched polysaccharides, such as arabinogalactans, for example, gum arabic, may also promote the exfoliation in water of carbon nanotubes. See Nano Lett. vol. 1, No. 12, 697-702, 2001.

The nanotubes may be present in the cosmetic composition in weight proportions ranging, for example, from 0.00001% to 30%, further, for example, from 0.0001% to 5%, and even further, for example, from 0.001% to 1% of the total weight of the composition.

The cosmetically acceptable medium of the solution comprising the nanotubes may, for example, be chosen from at least one of:

water,

aliphatic and aromatic alcohols, for example, ethanol, benzyl alcohol, fatty alcohols, modified and unmodified polyols such as glycerol, glycol, propylene glycol, dipropylene glycol, butylene glycol and butyl diglycol,

volatile and non-volatile silicones,

mineral, organic and plant oils,

oxyethylenated and non-oxyethylenated waxes, paraffins and alkanes, for example, C₅ to C₁₀ alkanes,

fatty acids, fatty amides, fatty esters, for example, fatty alkyl benzoates and salicylates, and

acetone, methyl ethyl ketone, methyl acetate, butyl acetate, ethyl acetate, dimethoxyethane and diethoxyethane.

The cosmetically acceptable medium may be in a form chosen from unmodified forms, emulsified forms, and encapsulated forms.

The compositions disclosed herein may also comprise at least one propellant. The at least one propellant may, for example, be chosen from compressed and liquefied gases usually used for preparing aerosol compositions. The at least one propellant may, for example, be chosen from compressed air, carbon dioxide, and nitrogen, soluble gases, for example, dimethyl ether, halo hydrocarbons (for example, fluoro hydrocarbons) and non-halo hydrocarbons.

The composition may further comprise at least one common cosmetic additive chosen from the following non-exhaustive list of additives: reducing agents, oxidizing agents, fatty substances, silicones, thickeners, softeners, antifoams, moisturizers, emollients, basifying agents, plasticizers, sunscreens, direct dyes and oxidation dyes, pigments, mineral fillers, clays, colloidal minerals, nacres, fragrances, peptizers, preserving agents, fixing and non-fixing polymers, conditioning polymers, proteins and vitamins.

After application to the hair, the composition may, for example, be rinsed out or left in. The composition may be in various forms, such as in a form chosen from lotions, sprays, mousses, shampoos and conditioners.

Also disclosed herein is the cosmetic use of nanotubes to volumize keratin fibers. The nanotubes may be as defined above.

The example that follows is intended to illustrate the embodiments disclosed herein without, however, being limiting in nature. The term “a.m.” means active material.

### EXAMPLE

Demonstration of the Long-Lasting Volumizing Effect given to Keratin Fibers

1 g of composition according to the embodiments disclosed herein and 1 g of control composition were applied to hair (2.7 g of European chestnut-brown hair about 20 cm long). After an action time of 2 minutes, the hair was rinsed with water and then shaped using a hairdryer.

Control composition

| | |
|---|---|
| Cyclopentasiloxane (1) | 10% a.m. |
| Oxyethylenated (7 EO) hydrogenated castor oil (2) | 10% a.m. |
| Dimethicone copolyol (3) | 0.5% a.m. |
| Propylene glycol | 2.5% a.m. |
| Behentrimonium chloride (4) | 1.2% a.m. |
| Water | qs. 100% |

Composition according to the disclosed embodiments

| | |
|---|---|
| Cyclopentasiloxane (1) | 10% a.m. |
| Oxyethylenated (7 EO) hydrogenated castor oil (2) | 10% a.m. |
| Dimethicone copolyol (3) | 0.5% a.m. |
| Propylene glycol | 2.5% a.m. |
| Behentrimonium chloride (4) | 1.2% a.m. |
| Single-wall carbon nanotubes (5) | 0.01% a.m. |
| Water | qs. 100% |

(1) DC 245 Fluid sold by the company Dow Corning
(2) Arlacel 989 sold by the company Uniquema
(3) DC 2-5225 C sold by the company Dow Corning
(4) Genamin KDM-F sold by the company Hoechst
(5) Single-wall Carbon Nanotubes of reference MRSW sold by the company MER Corporation.

The test was performed on 20 models, 10 models with application of the composition according to the disclosed embodiments and 10 models with application of the control composition. The cosmetic characteristics of the hairstyles were then evaluated by a panel of 10 individuals.

The panel unanimously indicated that the application of the composition according to the disclosed embodiments generated a marked effect of reduced bending of the fibers due to the effect of gravity. This phenomenon was reflected visually by detachment of the hairs at the root and a more pronounced provision of volume. After applying the composition according to the disclosed embodiments, the hairs were entirely individualized and their feel was very close to that of the hairs subjected to the control composition.

The tactile and visual evaluation of the various heads of hair was repeated according to the same process and after having successively performed two shampoo washes (with camomile DOP shampoo) followed by blow-drying.

The panel unanimously indicated that the cosmetic effects, and, for example, the volumizing effect imparted by using the composition comprising the carbon nanotubes, remained visible after two shampoo washes.

Demonstration of the Volumizing Effect

In order to demonstrate the effect of reduced bending of the keratin fibers under the effect of gravity after applying the composition according to the disclosed embodiments, the flexural rigidity of the fiber was measured by means of the “flexibility pendulum” method, before and after treating the fibers.

The pendulum used is of the rigid balance type which has a one-second beat. The pendulum consists of a bending bar made of polished brass. This bar is connected to an axis via a rod of length L=30 cm. On this rod, at a distance L=18.5 cm from the axis of rotation, is attached a heavy bar of mass m=47 g. The initial potential energy of the balance is set by its angle of inclination, written α.

The test samples of hair were in the form of a comb of 39 fragments of hair 11 mm long bonded in parallel onto a metal support.

The measurement was performed in the following manner: the brass bar was released from an angle α=30° with no initial speed. At each passage through the bottom point (α=0°), the bar bended the sample of 39 hairs and lost some of its potential energy, and did so until the pendulum came to a complete stop. The faster the pendulum stopped, the more rigid the hair. It was considered that the loss of energy associated with the friction of the bar on the hairs was negligible.

To demonstrate the rigidification of the fiber by applying the composition, a measurement was performed before treatment, followed by a second measurement after treatment. The number of beats required to stop the pendulum before and after treatment of the sample of hairs was then compared. The treatment of the hairs was performed directly on the hairs mounted on the test samples so as to obtain greater measurement sensitivity. The measurements and also the drying of the test samples of hair were performed at controlled temperature and relative humidity (20° C. and 45% RH).

Results: By applying the composition described in Example 1, and on the basis of 10 test samples of hair measured, it was observed that treatment of the hairs with the composition according to the embodiments disclosed induced an average 10%±2% reduction in the number of beats required to stop the pendulum. These results showed that the application of a composition comprising carbon nanotubes could make it possible to rigidify keratin fibers, which was reflected by the reduced bending of the fibers under the effect of gravity, visualized by a more pronounced volumizing of the hair.

What is claimed is:

1. A cosmetic composition comprising, in a cosmetically acceptable medium, nanotubes comprising at least one element chosen from elements of groups II_{A}, III_{A}, IV_{A}, V_{A}, VIII, I_{B}, II_{B}, III_{B}, VI_{B} and VII_{B} of the Periodic Table of the elements.

2. The composition according to claim 1, wherein the nanotubes comprise at least one element chosen from elements of group IV_{A} of the Periodic Table of the elements.

3. The composition according to claim 2, wherein the nanotubes comprise carbon as at least one element chosen from elements of group IV_{A} of the Periodic Table of the elements.

4. The composition according to claim 3, wherein the nanotubes totally or partially comprise organic molecules.

5. The composition according to claim 3, wherein the skeleton of the nanotubes comprises solely carbon atoms.

6. The composition according to claim 5, wherein the carbon nanotubes are single-wall nanotubes.

7. The composition according to claim 5, wherein the carbon nanotubes are multi-wall nanotubes.

8. The composition according to claim 5, wherein the surface of the carbon nanotubes is functionalized.

9. The composition according to claim 8, wherein the surface of the carbon nanotubes is functionalized by at least one reaction mechanism.

10. The composition according to claim 9, wherein the at least one reaction mechanism is chosen from nucleophilic substitution, electrophilic substitution, free-radical substitution, addition, elimination, rearrangement, oxidation, reduction, acid-base reaction, electrochemical reaction and photochemical reaction.

11. The composition according to claim 8, wherein the surface of the carbon nanotubes is functionalized with at least one functional group chosen from carboxylic groups, hydrophobic functional groups, fatty-chain amides, oligomers, polymers and dendrimers.

12. The composition according to claim 11, wherein the at least one functional group is capable of creating with keratin fibers at least one chemical bond chosen from Van der Waals interactions, hydrogen bonding, ionic bonds and covalent bonds.

13. The composition according to claim 12, wherein the at least one functional group capable of creating with keratin fibers at least one chemical bond chosen from covalent bonds is chosen from groups capable of reacting with thiols, disulphides, carboxylic acids, alcohols and amines.

14. The composition according to claim 13, wherein the at least one functional group capable of reacting with thiols, disulphides, carboxylic acids, alcohols and amines is chosen from:
epoxides,
groups comprising at least one aziridine ring,
vinyl and activated-vinyl groups,
carboxylic acids and derivatives thereof,
acetals and hemiacetals,
aminals and hemiaminals,
ketones, α-hydroxy ketones and α-halo ketones,
lactones and thiolactones,
isocyanate,
thiocyanate,
imines,
imides chosen from succinimides and glutimides,
imidates,
oxazine and oxazoline,
oxazinium and oxazolinium,
alkyl, aryl, and aralkyl halides, wherein the halogen is chosen from iodine, bromine and chlorine,
unsaturated-ring halides, wherein the unsaturated-rings are chosen from carbon-based rings and heterocycles,
sulphonyl halides of formula RSO₂X, wherein R is chosen from alkyl groups and X is chosen from fluorine and chlorine, and
silicon derivatives.

15. The composition according to claim 14, wherein the vinyl and activated vinyl groups are chosen from acrylonitrile, acrylic and methacrylic esters, crotonic acids and esters, cinnamic acids and esters, styrene and derivatives thereof, butadienes, vinyl ethers, vinyl ketones, maleic esters, maleimides, vinyl sulphones, and vinyl cyanoacrylates.

16. The composition according to claim 14, wherein the carboxylic acids and derivatives thereof are chosen from anyhydrides, and acid chloride and ester functional groups.

17. The composition according to claim 14, wherein the imides are N-hydroxysuccinimide ester.

18. The composition according to claim 14, wherein, in defining the unsaturated-ring halides, the heterocycles are chosen from chlorotriazines, chloropyrimidines, chloroquinoxalines, and chlorobenzotriazoles.

19. The composition according to claim 14, wherein the silicon derivatives are chosen from alkoxysilanes and silanols.

20. The composition according to claim 1, wherein the diameter of the nanotubes ranges from 1 to 300 nm and the length of the nanotubes ranges from 10 nm to 10 mm.

21. The composition according to claim 1, wherein the nanotubes are present in an amount ranging from 0.00001% to 30% by weight, relative to the total weight of the composition.

22. The composition according to claim 21, wherein the nanotubes are present in an amount ranging from 0.0001 % to 5% by weight, relative to the total weight of the composition.

23. The composition according to claim 22, wherein the nanotubes are present in an amount ranging from 0.001% to 1% by weight, relative to the total weight of the composition.

24. The composition according to claim 8, further comprising at least one surfactant.

25. The composition according to claim 24, wherein the at least one surfactant is chosen from amphiphilic molecules, amphiphilic oligomers, amphiphilic dendrimers and amphiphilic polymers.

26. The composition according to claim 24, wherein the at least one surfactant is capable of creating with a keratin material at least one covalent chemical bond by reaction with the thiol, disulphide, carboxylic acid, alcohol and amine functional groups of the amino acids of which the keratin material is composed.

27. The composition according to claim 26, wherein the at least one surfactant comprises at least one succinimidyl ester functional group.

28. The composition according to claim 1, wherein the cosmetically acceptable medium is chosen from at least one of:
water,
aliphatic and aromatic alcohols,
volatile and non-volatile silicones,
mineral, organic and plant oils,
oxyethylenated and non-oxyethylenated waxes, paraffins and alkanes,
fatty acids, fatty amides, and fatty esters, and
acetone, methyl ethyl ketone, methyl acetate, butyl acetate, ethyl acetate, dimethoxyethane and diethoxyethane.

29. The composition according to claim 28, wherein the aliphatic and aromatic alcohols are chosen from ethanol, benzyl alcohols, fatty alcohols, and modified and unmodified polyols.

30. The composition according to claim 29, wherein the modified and unmodified polyols are chosen from glycerol, glycol, propylene glycol, dipropylene glycol, butylene glycol and butyl diglycol.

31. The composition according to claim 28, wherein the alkanes are chosen from C₅ to C₁₀ alkanes.

32. The composition according to claim 28, wherein the fatty esters are chosen from fatty alkyl benzoates and salicylates.

33. The composition according to claim 1, wherein the cosmetically acceptable medium is in a form chosen from unmodified forms, emulsified forms, and encapsulated forms.

34. The composition according to claim 1, further comprising at least one propellant.

35. The composition according to claim 34, wherein the at least one propellant is chosen from compressed and liquefied gases chosen from compressed air, carbon dioxide, and nitrogen, soluble gases, halo hydrocarbons and non-halo hydrocarbons.

36. The composition according to claim 35, wherein the soluble gases are dimethyl ether.

37. The composition according to claim 35, wherein the halo hydrocarbons are chosen from fluoro hydrocarbons.

38. The composition according to claim 1, further comprising at least one cosmetic additive chosen from reducing agents, oxidizing agents, fatty substances, silicones, thickeners, softeners, antifoams, moisturizers, emollients, basifying agents, plasticizers, sunscreens, direct dyes, oxidation dyes, pigments, mineral fillers, clays, colloidal minerals, nacres, fragrances, peptizers, preserving agents, fixing and non-fixing polymers, conditioning polymers, proteins and vitamins.

39. The composition according to claim 1, wherein the composition is in a form chosen from a lotion, a spray, a mousse, a shampoo and a conditioner.

40. A method for volumizing keratin fibers comprising applying to the keratin fibers a cosmetic composition comprising, in a cosmetically acceptable medium, nanotubes.

41. The method according to claim 40, wherein the nanotubes comprise at least one element chosen from elements of groups II_{A}, III_{A}, IV_{A}, V_{A}, VIII, I_{B}, II_{B}, III_{B}, VI_{B} and VII_{B} of the Periodic Table of the elements.

42. The method according to claim 41, wherein the nanotubes comprise at least one element chosen from elements of group IV_{A} of the Periodic Table of the elements.

43. The method according to claim 42, wherein the nanotubes comprise carbon as at least one element chosen from elements of group IV_{A} of the Periodic Table of the elements.

44. The method according to claim 43, wherein the nanotubes totally or partially comprise organic molecules.

45. The method according to claim 43, wherein the skeleton of the nanotubes comprises solely carbon atoms.

46. The method according to claim 45, wherein the carbon nanotubes are single-wall nanotubes.

47. The method according to claim 45, wherein the carbon nanotubes are multi-wall nanotubes.

48. The method according to claim 45, wherein the surface of the carbon nanotubes is functionalized.

49. The method according to claim 48, wherein the surface of the carbon nanotubes is functionalized by at least one reaction mechanism.

50. The method according to claim 49, wherein the at least one reaction mechanism is chosen from nucleophilic substitution, electrophilic substitution, free-radical substitution, addition, elimination, rearrangement, oxidation, reduction, acid-base reaction, electrochemical reaction and photochemical reaction.

51. The method according to claim 48, wherein the surface of the carbon nanotubes is functionalized with at least one group chosen from carboxylic groups, hydrophobic functional groups, fatty-chain amides, oligomers, polymers and dendrimers.

52. The method according to claim 51, wherein the at least one functional group is capable of creating with the keratin fibers at least one chemical bond chosen from Van der Waals interactions, hydrogen bonding, ionic bonds and covalent bonds.

53. The method according to claim 52, wherein the at least one functional group capable of creating with the keratin fibers at least one chemical bond chosen from covalent chemical bonds is chosen from groups capable of reacting with thiols, disulphides, carboxylic acids, alcohols and amines.

54. The method according to claim 53, wherein the at least one functional group capable of reacting with thiols, disulphides, carboxylic acids, alcohols and amines is chosen from:
epoxides,
groups comprising at least one aziridine ring,
vinyl and activated-vinyl groups,
carboxylic acids and derivatives thereof,
acetals and hemiacetals,
aminals and hemiaminals,
ketones, α-hydroxy ketones and α-halo ketones,
lactones and thiolactones,
isocyanate,
thiocyanate,
imines,
imides chosen from succinimides and glutimides,
imidates,
oxazine and oxazoline,
oxazinium and oxazolinium,
alkyl, aryl, and aralkyl halides, wherein the halogen is chosen from iodine, bromine and chlorine,
unsaturated-ring halides, wherein the unsaturated rings are chosen from carbon-based rings and heterocycles,
sulphonyl halides of formula RSO₂X, wherein R is chosen from alkyl groups and X is chosen from fluorine and chlorine, and
silicon derivatives.

55. The method according to claim 54, wherein the vinyl and activated-vinyl groups are chosen from acrylonitrile, acrylic and methacrylic esters, crotonic acids and esters, cinnamic acids and esters, styrene and derivatives thereof, butadiene, vinyl ethers, vinyl ketones, maleic esters, maleimides, vinyl sulphones, and vinyl cyanoacrylates.

56. The method according to claim 54, wherein the carboxylic acids and derivatives thereof are chosen from anhydrides, and acid chloride and ester functional groups.

57. The method according to claim 54, wherein the imides are N-hydroxysuccinimide ester.

58. The method according to claim 54, wherein the heterocycles are chosen from chlorotriazines, chloropyrimidines, chloroquinoxalines, and chlorobenzotriazoles.

59. The method according to claim 54, wherein the silicone derivatives are chosen from alkoxysilanes and silanols.

60. The method according to claim 40, wherein the diameter of the nanotubes ranges from 1 to 300 nm and the length of the nanotubes ranges from 10 nm to 10 mm.

61. The method according to claim 40, wherein the keratin fibers are hair.

62. A cosmetic composition for volumizing keratin fibers comprising, in a cosmetically acceptable medium, nanotubes comprising at least one element chosen from elements of groups II_{A}, III_{A}, IV_{A}, V_{A}, VIII, I_{B}, II_{B}, III_{B}, VI_{B} and VII_{B} of the Periodic Table of the elements, wherein the nanotubes are present in the cosmetic composition in an effective amount to volumize the keratin fibers.

63. A process of manufacturing a cosmetic composition, comprising including in the composition nanotubes, wherein the composition is effective for volumizing keratin fibers.

64. The process according to claim 63, wherein the nanotubes comprise at least one element chosen from elements of groups II_{A}, III_{A}, IV_{A}, V_{A}, VIII, I_{B}, IIB, III_{B}, VI_{B} and VII_{B} of the Periodic Table of the elements.

65. The composition according to claim 1, wherein the nanotubes comprise at least one element chosen from carbon, silicon, tungsten, silver, gold, boron, zinc, platinum, magnesium, iron, cerium and aluminium.
